SEQUENCE LISTING (10 sequences)

SEQ ID NO: 1 - DNA, 19 bp, Homo Sapiens
cggctctcctcagctcctt

SEQ ID NO: 2 - DNA, 18 bp, Homo Sapiens
gtgcttcgctggctttgg

SEQ ID NO: 3 - DNA, 21 bp, Homo Sapiens
ccccaagactgttaaggtgtg

SEQ ID NO: 4 - DNA, 20 bp, Homo Sapiens
acttctgggtgatgcgagtg

SEQ ID NO: 5 - DNA, 23 bp, Homo Sapiens
cgcagctcagaacccttggaagc

SEQ ID NO: 6 - DNA, 19 bp, Homo Sapiens
acgcttgcatttgtgtcgg

SEQ ID NO: 7 - DNA, 23 bp, Homo Sapiens
cagccctgttcagacttctcaat

SEQ ID NO: 8 - DNA, 20 bp, Homo Sapiens
gacctgggaaagagggaaag

SEQ ID NO: 9 - DNA, 18 bp, Homo Sapiens
ggcgaaactccagcgaag

SEQ ID NO: 10 - DNA, 29 bp, Homo Sapiens
taaggagatagagatgggcgggcagtagg

(19) United States
(12) Patent Application Publication
(10) Pub. No.: US 2009/0004646 A1
(43) Pub. Date: Jan. 1, 2009

(54) Method for Quantifying Methylated Dna

(75) Inventors: Matthias Schuster, Berlin (DE); Philipp Schatz, Berlin (DE)

Correspondence Address: KRIEGSMAN & KRIEGSMAN, 30 TURNPIKE ROAD, SUITE 9, SOUTHBOROUGH, MA, 01772, US

(73) Assignee: EPIGENOMICS AG, Berlin (DE)

(21) Appl. No.: 11/629,743

(22) PCT Filed: Jun. 15, 2005
(86) PCT No.: PCT/DE05/01109

§ 371 Date: Jul. 9, 2007

(30) Foreign Application Priority Data
Jun. 15, 2004 (DE) 10 2004 029 700.2

(51) Int. Cl. C12Q 1/68

(52) U.S. Cl. 435/6

(57) ABSTRACT

The inventive method makes it possible to quantify methylated DNA by combining the restricted digestion and real-time PCR. For this purpose, the inventive method consists in isolating the examined DNA from a biological sample, in reacting the isolated AND with a methylation specific restriction enzyme, in amplifying by means of a real-time PCR, wherein the amplified products are formed only when the DNA is precut-off and, afterwards, in calculated the methylated and unmethylated DNA proportion in the initial sample with the aid of a reference measurement. Said method is particularly suitable for diagnosis and prognosis cancer and other diseases associated with a methylation state modification and for predicting the drug effects.

### BACKGROUND OF THE INVENTION

The present invention relates to a method for quantifying methylated cytosine positions in DNA. 5-Methylcytosine is the most frequent covalently modified base in the DNA of eukaryotic cells. It plays an important biological role, among other things, in the regulation of transcription, in genetic imprinting and in tumorigenesis (for review: Millar et al.: Five not four: History and significance of the fifth base. In.: Epigenome, S. Beck and A. Olek (eds.), Wiley-VCH Publishers, Weinheim 2003, pp. 3-20). The identification of 5-methylcytosine is of considerable interest, particularly for cancer diagnosis. A detection of methylcytosine is difficult, of course, since cytosine and methylcytosine have the same base-pairing behavior. The conventional DNA analytical methods based on hybridization therefore cannot be applied. Accordingly, the usual methods for methylation analysis operate according to two different principles. In the first method, methylation-specific restriction enzymes are used, and in the second one, there occurs a selective chemical conversion of unmethylated cytosines to uracil (so-called: bisulfite treatment, see, e.g.: DE 101 54 317 A1; DE 100 29 915 A1). The DNA that has been pretreated enzymatically or chemically is then amplified for the most part and can be analyzed in different ways (for review: WO 02/072880 pp. 1 ff; Fraga and Esteller: DNA methylation: a profile of methods and applications. Biotechniques. 2002 September; 33(3): 632, 634, 636-49.). For sensitive analysis, the DNA is usually bisulfited and then amplified by means of different PCR methods (see: e.g., Herman et al.: Methylation-specific PCR: a novel PCR assay for methylation status of CpG islands. Proc. Natl. Acad. Sci. USA. 1996 Sep. 3; 93(18): 9821-6; Cottrell et al.: A real-time PCR assay for DNA methylation using methylation-specific blockers. Nucl. Acids Res. 2004 32: e10). Real-time PCR variants are used also (e.g.: “MethyLight”; for review: Trinh et al.: DNA methylation analysis by MethyLight technology. Methods. 2001 December; 25(4): 456-62); WO 00/70090; U.S. Pat. No. 6,331,393).

A quantification of the degree of methylation is necessary for various applications, e.g., for classifications of tumors, for prognostic determinations or for predicting drug actions. Different methods are known for quantification of the degree of methylation. As a rule, a chemical conversion of the DNA is performed first, followed by an amplification, e.g., by Ms-SnuPE, by hybridizations on microarrays, by hybridization assays in solution or by direct bisulfite sequencing (for review: Fraga and Esteller 2002, oc. cit.). One problem with these “end-point analyses” consists of the fact that the amplification may occur non-uniformly, among other things, due to product inhibition, enzyme instability and a decrease in the concentration of the reaction components. A correlation between the quantity of amplificate and the quantity of DNA utilized thus does not always result. The quantification is thus sensitive to error (see: Kains: The PCR plateau phase-towards an understanding of its limitations. Biochem. Biophys. Acta 1494 (2000) 23-27). In contrast, threshold-value analysis based on real-time PCR determines the quantity of amplificate, not at the end of the amplification, but in the exponential amplification phase. This method presumes that the amplification efficiency is constant in the exponential phase. The so-called threshold value Ct (cycle threshold) is a measure for that PCR cycle in which the signal in the exponential phase of the amplifications is for the first time greater than the background noise. The absolute quantification is then a result of a comparison of the Ct value of the investigated DNA with the Ct value of a standard (see: Trinh et al. 2001, loc. cit.; Lehmann et al.: Quantitative assessment of promoter hypermethylation during breast cancer development. Am J. Pathol. 2002 February; 160(2): 605-12).

A fundamental problem of the above-described quantification methods consists of the fact that the bisulfite conversion leads to large losses in yield and to damage of the DNA, e.g., by fragmenting (for review: Grunau et al.: Bisulfite genomic sequencing: systematic investigation of critical experimental parameters. Nucleic Acids Res. 2001 Jul. 1; 29(13)). In addition, it is difficult to remove the bisulfite salts from the reaction solution after the conversion, so that the DNA polymerase may be inhibited in the subsequent amplification. An additional problem resides in the fact that most clinical specimens are embedded in paraffin (paraffin embedded tissue, PET). In such specimen material, the DNA is already greatly fragmented prior to the bisulfite treatment, so that an analysis after a bisulfite conversion is even more difficult.

The above-described difficulties can be circumvented if a high-performing combination of restriction digestion and real-time PCR is successfully provided. A quantification assay based on restriction and real-time PCR has already been described for methylated DNA (Lebrun et al.: A methyltransferase targeting assay reveals silencer-telomere interactions in budding yeast. Mol. Cell Biol. 2003 March; pages 1498-1508, in particular, page 1500, FIG. 1b; Chu et al.: The use of real-time quantitative polymerase chain reaction to detect hypermethylation of the CpG islands in the promoter region flanking the GSTP1 gene to diagnose prostate carcinoma. J. Urol. 2002 April; 167(4): 1854-8). In this method, a fragment is amplified, which comprises a recognition site for a restriction enzyme. Prior to the amplification, the DNA to be investigated is reacted with a restriction enzyme, which cleaves the unmethylated, but not the methylated DNA. In the subsequent PCR, only the uncleaved-methylated—DNA can be amplified. For a quantification according to this method, however, it is necessary that the total quantity of DNA utilized is determined first. Only in this way is it possible to assign an actual value to the signals that are produced. The determination of the DNA concentration, however, requires an additional operating step and thus creates an additional source of error. A determination of the DNA concentration according to conventional methods is particularly difficult in paraffin-embedded tissue. Often, only a portion of this DNA can actually be amplified due to irreversible modifications.

Another combination of restriction digestion and real-time PCR is described in Fukuzawa et al. (Epigenetic differences between Wilms' tumours in white and east Asian children. Lancet. 2004 Feb. 7; 363(9407): 446-51). This method is utilized for the investigation of imprinting. A sensitive quantification is not conducted.

A combination of restriction digestion and real-time PCR that makes possible a sensitive quantification in a surprisingly simple way, even without a determination of the DNA concentration will be described for the first time in the following. Experimental set-up, conducting and evaluation of the quantitative methylation analysis are thus clearly simplified. Based on the particular biological and medical importance of cytosine methylation and due to the above-mentioned disadvantages of the prior quantification methods, the method according to the invention represents an important technical advance.

### DESCRIPTION

The invention involves a method for quantifying methylated DNA, in which the following steps are conducted:
a) the DNA is converted with at least one methylation-specific restriction enzyme,
b) a real-time PCR is conducted, wherein the sequence to be investigated is only amplified if it has not been cleaved previously in step a),
c) the fraction of methylated DNA in the original specimen is calculated from the signal of the sequence to be investigated and the signal of a reference measurement.

In the first step of the method according to the invention, the DNA to be investigated is made accessible to the restriction enzymes or is isolated. The DNA thus can originate from different sources depending on the diagnostic or scientific objective. For diagnostic objectives, tissue samples preferably serve as the initial material. As described above, the application of the method according to the invention has particular advantages for the analysis of paraffin-embedded specimens. In addition, however, it is also preferred to analyze the DNA from body fluids, particularly serum. Here, an isolation of the DNA is not necessary in every case. It is also conceivable to use the DNA from sputum, stool, urine, or cerebrospinal fluid. The DNA is isolated according to standard methods, from blood, e.g., with the use of the Qiagen UltraSens DNA extraction kit.

In the second step of the method according to the invention, the DNA is converted with at least one methylation-specific restriction enzyme. Here, at least one restriction site lies within the sequence that will be amplified in the third step. It is therefore achieved that only the DNA of one methylation state is amplified.

The restriction is preferably conducted with enzymes that specifically cleave unmethylated DNA, so that only the methylated DNA is amplified. Insofar as they may be available, however, enzymes that specifically cleave methylated DNA (e.g., McrB, New England Biolabs, USA) can also be utilized. There is a large number of restriction enzymes that can be used for the method according to the invention. More detailed information on restriction enzymes can be found, e.g., in the database “Rebase” (http://rebase.neb.com/; see also: Roberts et al.: REBASE: restriction enzymes and methyltransferases. Nucleic Acids Research, 2003, Vol. 31, No. 1, 418-420).

In a preferred embodiment of the invention, several restriction cleavage sites lie within the sequence to be amplified. Thus, the probability that the fragment will be cleaved is increased. The danger of false-positive signals is therefore reduced. This applies particularly to the analysis of DNA from paraffin specimens. Particularly preferred, the sequence to be amplified bears up to five cleavage sites. First of all, it is possible that the sequence contains several restriction cleavage sites for the same enzyme. In addition, it is also conceivable that the sequence bears several restriction cleavage sites for different enzymes. The restriction then takes place in parallel simultaneously with several enzymes, whereby the different enzymes should be active under the same reaction conditions. Alternatively, the restriction will be carried out sequentially.

Depending on the enzyme utilized, the restriction takes place according to standard conditions. These conditions are to be found, e.g., in the protocols supplied by the manufacturers.

In the third step of the method according to the invention, a real-time PCR is conducted, wherein the sequence to be investigated will only be amplified if it has not been cleaved previously in the second step.

A real-time PCR in the following will be understood as a PCR which permits detection of the amplificates even during the amplification. Different real-time PCR variants are used by the person skilled in the art, e.g., SYBR-Green, Lightcycler, Taqman, Sunrise, Molecular Beacon or Eclipse forms. Details for the construction of the primers and the probes belong to the prior art (refer to: U.S. Pat. No. 6,331,393 with further citations). Thus, the design of the probes can be carried out, e.g., via “Primer Express” software of Applied Biosystems (for Taqman probes) or via MGB Eclipse Design Software of Epoch Biosciences (for Eclipse probes).

The PCR is thus designed such that the amplificates span the restriction sites. Therefore, amplificates will be formed only if a restriction has not occurred previously.

In a preferred embodiment of this invention, the amplificates are selected such that they are between 50 bp and 150 bp long. In this way, fragmented DNA from paraffin-embedded tissue can be more easily investigated.

In the fourth step of the method according to the invention, the fraction of methylated DNA in the original specimen is calculated from the signal of the sequence to be investigated and the signal of a reference measurement. The reference measurement may be made also before the restriction, so that each reaction contains the same quantity of DNA. The quantity of DNA detected after the restriction (e.g., methylated DNA) is then referred to the quantity utilized (total DNA). The total DNA can be determined according to known methods, e.g., by means of a real-time PCR. The degree of methylation of the sample can then be calculated from this ratio. Alternatively, a portion of the DNA to be investigated will not be treated with enzymes and the DNA quantity of this DNA will be quantified in parallel in a second reaction with the same fragment (see details below). In a particularly preferred embodiment, a reference fragment is used.

The reference fragment involves a sequence that represents the total DNA in the reaction batch, independent of the methylation state. The reference fragment is not cleaved by the restriction enzymes and thus is amplified in the PCR independent of methylation state. This can be achieved in one embodiment due to the fact that the reference fragment does not have cleavage sites for the restriction enzymes utilized. The fraction of methylated DNA can be quantified by a comparison of the signals of the total DNA and the methylated DNA. The reference fragment is preferably approximately the same size as the sequence to be analyzed and can be amplified with equal efficiency. Also, it is preferably located in the vicinity, most preferably in the direct vicinity of the sequence to be analyzed. It is assured in this way that fragmentations of the DNA, as they frequently occur in paraffin specimens, do not lead to false results. In a particularly preferred embodiment of the method according to the invention, the reference fragment is identical to the fragment to be analyzed. The reaction batch is then divided up and only a portion is digested (see below).

In a preferred embodiment of the method according to the invention, the amplifications of the sequence to be analyzed and of the reference fragment take place simultaneously in one reaction vessel. This has the advantage that the reaction conditions are identical for both fragments. In this embodiment, it is necessary, of course, that the two probes bear different labels.

In another preferred embodiment, the amplifications take place in different vessels. In this way, disruptive interactions between the fluorescent dyes can be avoided and a competition between the two amplifications is excluded.

Finally, the degree of methylation of the sequence to be analyzed can be quantified by means of the signals of the reference fragment. Signal detection is made according to the real-time variant of the prior art that is utilized in each case. Quantification can be carried out via different calculation methods. Preferably, the difference in the two Ct values (threshold values) is utilized as the quantitative criterion for the degree of methylation. As described above, the Ct value is a measure for that PCR cycle in which the signal in the exponential phase of the amplification is for the first time greater than the background noise. According to the invention, the quantification is performed based on the following principle: one uses enzymes that specifically cleave unmethylated DNA, so that only the methylated DNA is amplified. The smaller the difference is between the Ct of the reference fragment (amplification of the total DNA) and the Ct of the digested specimen to be analyzed (amplification only of the methylated DNA), the greater is the fraction of methylated DNA.

The degree of methylation M can be calculated via the ratio of the DNA quantities determined by two independent DNA quantifications. The first quantification is carried out with the fragment to be investigated, so that only the quantity of the uncleaved DNA is determined. The second quantification can be carried out selectively with a second reference fragment whose sequence does not contain cleavage sites or with the fragment to be investigated by investigating the uncleaved DNA sample. This reference DNA may be an aliquot of the DNA to be analyzed, which was not treated with restriction enzymes. In the simplest case, the threshold value (Ct) is determined for each quantification and the DNA quantity is obtained via comparison with a series of DNA quantification standards. This value is made into a ratio with the DNA quantity of the reference measurement.

M=DNA quantity_{fragment}/DNA quantity_{reference}.

In other cases, the degree of methylation M can be determined approximately according to the following formula:

M=E^{-ΔΔCt}

E is the PCR efficiency which amounts to 2 in the optimal case. ΔCt corresponds to the systematic difference between the threshold value of the uncleaved fragment that is investigated and the reference fragment for the identical quantity of DNA utilized. This difference is a constant to be determined individually for each fragment to be investigated. ΔΔCt is the difference between the threshold value of the restricted fragment that is investigated and the reference fragment minus the systematic difference ΔCt. For simplification, it is also possible to use the following formula:

M=2^{-ΔΔCt}

It results from this that the PCR is conducted with optimal efficiency.

If, instead of a reference fragment, the uncleaved specimen is compared with the restricted specimen, the amplification occurs only of the investigated fragment and the systematic difference cancels out. In this case, the degree of methylation is calculated according to the following formula:

M=E^{-ΔCt}

wherein here ΔCt corresponds to the difference between the threshold values of the restricted and non-restricted specimens.

For simplification, it is also possible to use the following formula:

M=2^{-ΔCt}

It results from this that the PCR is conducted with optimal efficiency.

A quantification via the above-described method is particularly well possible if the assay conditions have been optimized beforehand with respect to the PCR efficiency. This can be done by means of varying the primers, the probes, the temperature program and the other reaction parameters. The PCR efficiency can be determined via standard methods, e.g. by means of a standard of uncleaved genomic DNA.

When a reference fragment is used, the degree of methylation can also be determined with the use of standard DNA dilutions, as are commonly used in quantitative PCR. For this purpose, both for the investigated fragment as well as also for the reference fragment, standard amplification curves are determined and used for the purpose of converting the threshold values for both fragments into DNA quantities. In this way, depending on the restriction produced, the quantity of methylated DNA in the specimen will be determined from the threshold value of the investigated fragment and the total quantity of DNA in the specimen will be determined from the threshold value of the reference fragment. The ratio of the two values corresponds to the degree of methylation of the investigated fragment in the specimen.

The quantification can be calibrated with the use of a methylation standard (e.g., with 0%, 5%, 10%, 25%, 50%, 75% and 100% degree of methylation). The preparation of methylation standards and their application in methylation analysis is described in detail in the European Patent Application with the reference EP 04 090 037.5 (Applicant: Epigenomics AG).

A particularly preferred embodiment of the method according to the invention uses controls, with which an examination can be made of whether the restriction is complete. This can be done with different variants. In one embodiment, unmethylated DNA is used as a control. The unmethylated DNA should be completely converted by the restriction enzymes, so that an amplificate is not formed in the PCR. Unmethylated DNA is available from different sources, e.g., a genome-wide amplification method (refer to European Patent Application 04 090 037. 5, date filed: Feb. 5, 2004, Applicant: Epigenomics AG). Another embodiment assures that the restriction enzymes will not be inhibited by components of the biological specimen. Thus, another restriction enzyme can be utilized as a control, which cleaves independent of methylation within the fragment to be amplified. In this control, the total DNA should be digested independent of its methylation state. The PCR therefore should not generate an amplificate. It is particularly preferred in this variant to utilize isoschizomers, which provide the same recognition sequence as the other restriction enzymes utilized, but cleave independent of methylation. In a third control variant, a control gene which is never present in methylated form is analyzed in parallel with the fragment to be analyzed. The corresponding DNA should be completely cleaved in the restriction, so that here also an amplificate should not be formed in the PCR. In this way, problems can be addressed, such as, e.g., the inappropriate preparation of paraffin specimens.

In a particularly preferred embodiment of the method according to the invention, the sequence to be analyzed and the reference fragment are identical. In this case, the specimen to be investigated is divided and only a portion of the specimen is reacted with restriction enzymes. The other portion remains untreated.

Accordingly, the invention is a method for quantifying methylated DNA, in which the following steps are conducted:
a) the DNA is divided into two equal portions,
b) the first portion of the DNA is reacted with a methylation-specific restriction enzyme, while the other portion remains untreated,
c) the DNA of both portions is amplified by means of a real-time PCR, wherein fragments will only be formed in the first portion of the specimen if the DNA has not been cleaved previously,
d) the fractions of methylated and unmethylated DNA in the original specimen are calculated from the signals of the two portions.

In the first step of the method according to the invention, the DNA to be investigated, from different sources as described above, is made accessible to the restriction enzymes or is isolated. In a particularly preferred embodiment, paraffin specimens are investigated.

In the second step of the method according to the invention, the isolated DNA is divided into two equal portions. One of the specimen portions is then reacted with a methylation-specific restriction enzyme in the third step. In this case, at least one restriction site lies within the sequence that will be amplified in the fourth step. It is therefore achieved that only the DNA of one methylation state is amplified. In contrast, the second reaction batch is not reacted with a restriction enzyme. In this case, the total DNA is then amplified independent of the methylation state in the subsequent PCR.

The restriction is preferably conducted with enzymes that specifically cleave unmethylated DNA, so that only the methylated DNA is amplified. Insofar as they may be available, however, enzymes that specifically cleave methylated DNA (see above) can also be utilized. As described above in detail, there is a multiplicity of restriction enzymes that can be used for the method according to the invention.

In a preferred embodiment of the invention, several restriction cleavage sites lie within the sequence to be amplified (see above). Thus, the probability that the fragment will be cleaved is increased. The danger of false-positive signals is therefore reduced. This applies particularly to the analysis of DNA from paraffin specimens. Particularly preferred, the sequence to be amplified bears up to five cleavage sites. First of all, it is possible that the sequence contains several restriction cleavage sites of the same enzyme. In addition, it is also conceivable that the sequence bears several restriction cleavage sites of different enzymes. The restriction then takes place in parallel simultaneously with several enzymes, whereby the different enzymes should be active under the same reaction conditions.

The restriction takes place dependent on the enzyme utilized according to standard conditions. These conditions are to be found, e.g., in the protocols supplied by the manufacturers. It is important to add to the second reaction batch, in which a restriction has not occurred, the same reagents that are also contained in the first batch. In this way, it can be achieved that the subsequent amplification takes place under reaction conditions that are as identical as possible.

In the fourth step of the method according to the invention, both reaction batches are amplified by means of a real-time PCR (see above). The PCR is designed in such a way that the amplificates span the restriction sites. Therefore, amplificates will be formed only if a restriction has not occurred previously.

In the fourth step of the method according to the invention, the fractions of methylated DNA and unmethylated DNA in the original specimen are calculated from the signals obtained from the two specimen portions that were separated beforehand. Signal detection is produced here according to the real-time variant of the prior art that is utilized in each case. The quantification can be conducted by means of different calculation methods, as described in detail above. Preferably, the difference in the two Ct values (threshold values) is utilized as the quantitative criterion for the degree of methylation. The quantification is performed according to the invention based on the following principle: One uses enzymes that specifically cleave the unmethylated state, so that only the methylated DNA is amplified. The smaller the difference is between the Ct of the undigested specimen (amplification of the total DNA) and the Ct of the digested specimen (amplification only of the methylated DNA), the greater is the fraction of methylated DNA. Possible formulas for calculating the degree of methylation are indicated above. In particular, the following formula can be used: M=E^{-ΔCt}

A quantification via the above-described method is particularly well possible if the assay conditions have been optimized beforehand with respect to the PCR efficiency (see above). Different control systems can be utilized, which are described in detail above and which are also applicable to this embodiment.

The method according to the invention is particularly well suitable for the sensitive quantification of degrees of methylation, The method according to the invention is particularly suitable for detecting the DNA of one methylation state (e.g., methylated DNA) against an intense background of DNA of the other methylation state (e.g.: unmethylated DNA). Thus, the fraction of DNA to be detected preferably amounts to less than 10%. In other preferred embodiments, the fraction of DNA to be detected amounts to less than 5%, or less than 1%.

Clinical specimens are particularly preferably investigated, in particular body fluids or tissue embedded in paraffin. A particularly preferred use of the method according to the invention lies in the diagnosis or prognosis of cancer diseases or other diseases associated with a modification of the methylation state. These include, among others, CNS malfunctions; symptoms of aggression or behavioral disturbances; clinical, psychological and social consequences of brain damage; psychotic disturbances and personality disorders; dementia and/or associated syndromes; cardiovascular diseases, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disease of the skin, the muscles, the connective tissue or the bones; endocrine and metabolic malfunction, damage or disease; headaches or sexual malfunction. The method according to the invention is also suitable for predicting undesired drug effects and for distinguishing cell types or tissues or for investigating cell differentiation.

The invention is also a kit for conducting the method according to the invention, which is comprised of at least one restriction enzyme, two primers, a polymerase as well as either a sequence-specific real-time probe or a non-sequence-specific intercalating fluorescent dye, as well as other optional reagents necessary for a PCR.

### EXAMPLES

### Example 1

### Investigation of Paraffin Specimens

The following example clarifies the invention. The methylation of the RASSF1 gene should be quantified in lung tumor specimens. For this purpose, DNA was prepared from paraffin-embedded sections of 10 lung tumor specimens and 10 normal tissue samples (normal adjacent lung tissue). The tissue was deparaffinized and lysed according to standard procedures. The DNA was extracted from the lysis buffer by means of the QIAmp DNA Mini Kit (Qiagen) and quantified by means of a UC measurement.

The following steps were conducted in parallel for all 20 samples. 2 μg of the DNA were dissolved in 380 μl of Y+/Tango buffer (Fermentas) in 0.5 ml PCR wells. 190 μl of this solution, precisely half of the total volume, were pipetted into an identical second PCR well. To the first batch was added 2.5 μl of HpaII und 2.5 μl of Hin6I restriction endonucleases (both Fermentas). To the second batch was added 5 μl of a mixture of glycerin and water (v:v=1:1). All 40 specimens were incubated at 37° C. in an Eppendorf Thermocycler for five hours. Subsequently, at another time, a mixture of restriction enzymes (or a mixture of water and glycerin) was added to the individual batches. The incubation was extended to 10 hours. After this time, the restriction enzymes were inactivated by heat (65° C. for 10 minutes).

Subsequently, 38 μl of each batch were pipetted into a 96-well plate and reacted with 62 μl of water. Finally, both the specimens treated with restriction enzymes as well as the untreated specimens were analyzed by means of a quantitative real-time PCR. For this purpose, 12 μl of a Master mix, which contained SybrGreen and the primers CGGCTCTCCTCAGCTCCTT (SEQ ID NO 1) and GTGCTTCGCTGGCTTTGG (SEQ ID NO 2) were pipetted into a 96-well Taqman plate. Then, 8 μl of the samples were added each time (as a double value each time). The real-time PCR was conducted in an ABI 7700 device with the following temperature profile: 15 minutes at 95° C.; 50 cycles of: 15 seconds at 95° C., 45 seconds at 65° C., 75 seconds at 72° C.; 15 seconds at 95° C.; followed by one hour at 50° C.

Only one amplification of the methylated copies of the RASSF1 gene was observed in the enzyme-treated batch, since the unmethylated copies were digested prior to the PCR. In contrast, both methylated as well as unmethylated DNA were amplified in the undigested batch. The equation

ΔCt=Ct_{blank}−Ct_{restricted}

describes the relative quantity of unmethylated DNA in a DNA specimen. A ΔCt value of zero means that the investigated DNA was completely methylated. A value of 0.5 represents a 50% methylation in the case of optimized PCR reaction conditions. Lower degrees of methylation lead to higher ΔCt values. The relative quantity of methylated RASSF1 gene in a specimen is described by the following equation:

M=E^{-ΔCt}

Here, E represents the PCR efficiency, which can be determined easily via standard procedures (see above).

FIG. 1 shows the ΔCt values for 20 samples embedded in paraffin (RAS SF. 1 lung samples 1-20), which were investigated in this experiment; FIG. 2 shows the corresponding rates of methylation for the assumed PCR efficiency of E=2. It can be seen from the figure that 5 of 10 tumor specimens, but only one of ten normal reference tissue samples, have an RASSF1 methylation of more than one percent. This result corresponds to the expectation that the investigated region is present in hypermethylated form in lung tumor tissue.

### Example 2

### Calibration by Means of an Untreated Reference Specimen

DNA from “buffy coat” DNA (lymphocytes) was mixed with DNA treated with SssI methyltransferase. The investigated promoter of the FOXL2 gene is not methylated in healthy lymphocytes. DNA mixtures were prepared with different levels of methylation (100%; 75%; 50%; 25%; 10%; 5%; 2.5%; 1.25%; 0.625%; 0.3125%; 0.156%; 0.078%; 0.039%; 0.019% and 0%). These were mixed with restriction buffer (Y-Tango buffer) and divided into two portions. One portion was cleaved with the methylation-sensitive enzymes HpaII and Hin6I. The other portion was not treated. The DNA quantity was quantified with a 5′-exonuclease method (TaqMan). The PCR was conducted under standard conditions with the following temperature program: 10 min 95° C., 45 cycles: 15 seconds 95° C., 60 seconds 65° C. A fragment with a length of 141 bp was amplified. The following were used as primers: forward primer: ccccaagactgttaaggtgtg (Seq ID 3); reverse primer: acttctgggtgatgcgagtg (Seq ID 4); Taqman probe: cgcagctcagaacccttggaagc (Seq ID 5).

The quantity of methylated DNA utilized in percent is plotted on the x-axis in FIG. 3. The ratio of the two quantification reactions is plotted on the y-axis (2^{-ΔCt}). The error bars correspond to the standard deviation of four repetitions. The level of methylation is logarithmically plotted on the x-axis in FIG. 4. The y-axis represents the ΔCts.

FIG. 5 lists the calculated Fisher scores for distinguishing the investigated level of methylation. A Fisher score of >2 permits such distinguishing. Values of less than 2 are characterized with an asterisk (*) in FIG. 5.

### Example 3

### Calibration by the Use of Equal DNA Quantities

Different DNA mixtures were prepared as in Example 2. Prior to the treatment with restriction enzymes, all specimens were quantified and equal DNA quantities were utilized each time. The degree of methylation can be determined from the ratio of the total quantity of DNA to the DNA quantity that was measured after the restriction.

The mean value for the Ct values is plotted vs. the level of methylation in FIG. 6.

FIG. 7 lists the calculated Fisher scores for distinguishing the investigated level of methylation. A Fisher score of >2 permits such distinguishing. Values of less than 2 are characterized with an asterisk (*) in FIG. 7.

### Example 4

### Calibration by Means of a Second Fragment in a Second Reaction

The degree of methylation of a sequence of the GSTPi gene will be investigated in specimens from prostate cancer patients. For this purpose, a control fragment that is not cleaved by the restriction enzymes utilized was used in one experiment. The control fragment also lies within the GSTPi and has a length of 135 bp. In another experiment, the sequence to be investigated was divided into two batches, only one of which was reacted with restriction enzymes. The amplificate has a length of 153 bp. The PCR was carried out under standard conditions with a SYBR® Green Master Mix with the following temperature program: 10 min 95° C.; 45 cycles (15 sec 95° C.; 45 sec 65° C.; 1:15 min 72° C.). The following primers were used: Control fragment: forward primer: acgcttgcatttgtgtcgg (Seq ID 6); reverse primer: cagccctgttcagacttctcaat (Seq ID 7). Fragment to be analyzed: forward primer: gacctgggaaagagggaaag (Seq ID 8); reverse primer: ggcgaaactccagcgaag (Seq ID 9).

The results are shown in FIG. 8. It is shown that both methods arrive at the same results. At the top, the normalization of the data was carried out by means of an external quantitative real-time PCR. At the bottom, the results are shown by means of an undigested control.

### Example 5

### Calibration by Means of a Second Fragment in a Duplex Reaction

The FOXL2 gene will be analyzed as in Example 2. For this purpose, DNA mixtures are prepared analogously to Example 1. Subsequently, the cleaved fraction will be quantified exclusively. For this purpose, a fragment whose sequence contains cleavage sites for the enzymes used, and a fragment without cleavage sites will be amplified in one reaction. The PCR is carried out under standard conditions with the following temperature program: 10 min 95° C., 45 cycles: 15 seconds 95° C., 60 seconds 65° C. The following are used as primers: forward primer: ccccaagactgttaaggtgtg (Seq ID 3); reverse primer: acttctgggtgatgcgagtg (Seq ID 4); Taqman probe: cgcagctcagaacccttggaagc (Seq ID 5); Control fragment forward primer: acgcttgcatttgtgtcgg (Seq ID 6); control fragment reverse primer: cagccctgttcagacttctcaat (Seq ID 7); control fragment Taqman probe: taaggagatagagatgggcgggcagtagg (Seq ID 10).

The quantity of the DNA utilized can be determined by means of the fragment without cleavage sites. The methylation of the sequence can be detected in the other fragment. In this way, the quantity of DNA utilized can be reduced and the variability in the quantification can be reduced as well.

### DESCRIPTION OF THE FIGURES

FIG. 1 shows the results of Example 1. Shown are the ΔCt values for 20 samples embedded in paraffin, which were investigated in this experiment.

FIG. 2 shows the results of Example 1. Shown are the methylation rates for an assumed PCR efficiency of E=2. It can be seen from the figure that 5 of 10 tumor specimens, but only one of ten normal reference tissue samples, have an RASSF1 methylation of more than one percent. This result corresponds to the expectation that the investigated region is present in hypermethylated form in lung tumor tissue.

FIG. 3 shows the results of Example 2. The quantity of methylated DNA utilized is plotted in percent on the x-axis. The ratio of the two quantification reactions is plotted on the y-axis (2^{-ΔCt}). The error bars correspond to the standard deviation of four repetitions.

FIG. 4 also shows the results of Example 2 (refer to FIG. 3). The level of methylation is logarithmically plotted on the x-axis. The y-axis represents the ΔCts.

FIG. 5 shows additional results of Example 2 (refer to FIGS. 3 and 4). Shown are the Fisher scores for distinguishing the investigated level of methylation. A Fisher score of >2 permits such distinguishing. Values of less than 2 are characterized with an asterisk (*) in FIG. 5.

FIG. 6 shows the results of Example 3. The mean value for the Ct values is plotted vs. the level of methylation.

FIG. 7 also shows the results of Example 3. Shown are the Fisher scores for distinguishing the investigated level of methylation. A Fisher score of >2 permits such distinguishing. Values of less than 2 are characterized with an asterisk (*) in FIG. 7.

FIG. 8 shows the results of Example 4. At the top, the normalization of the data was carried out by means of an external quantitative real-time PCR. At the bottom, the results are shown by means of an undigested control. It is shown that both methods arrive at the same results.

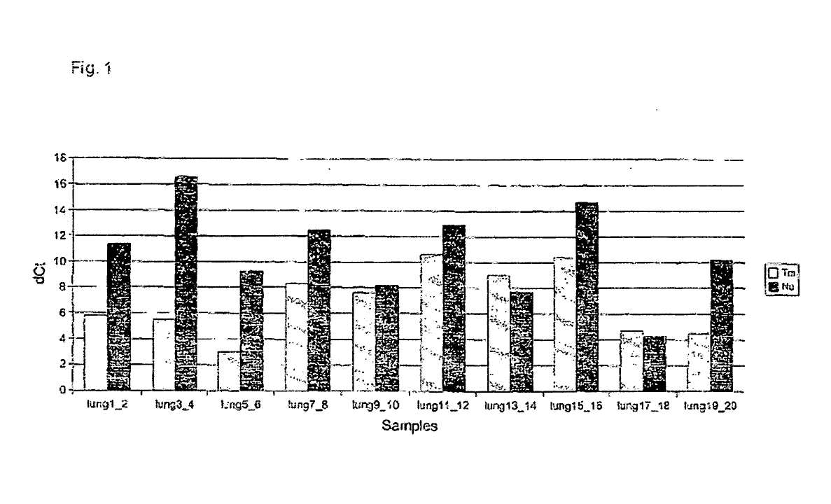

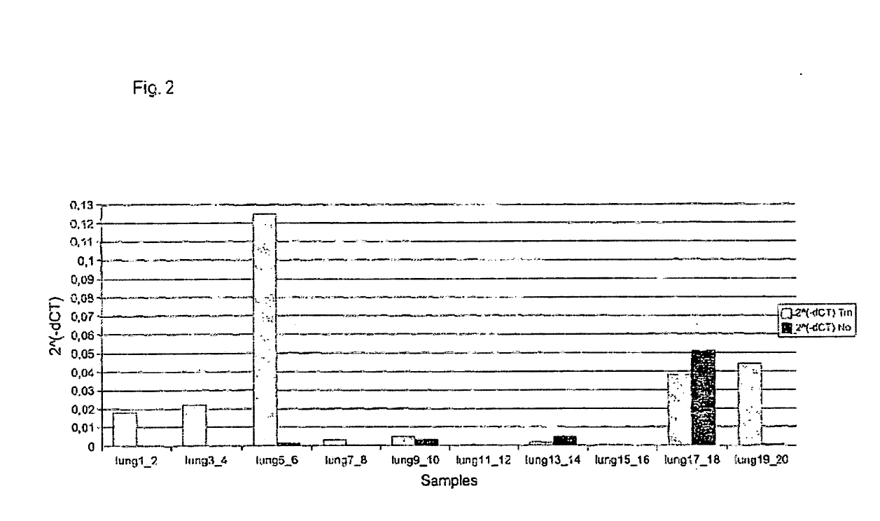

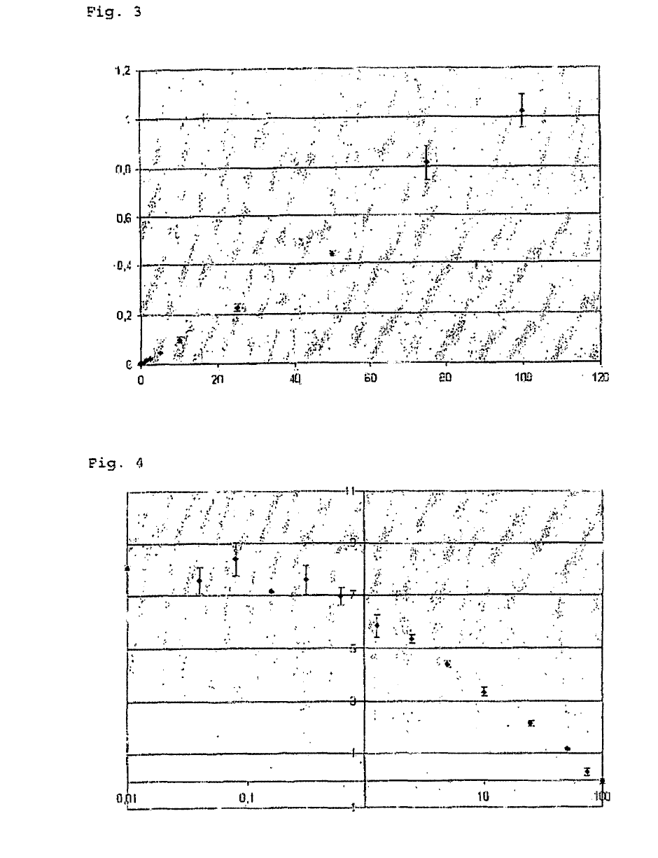

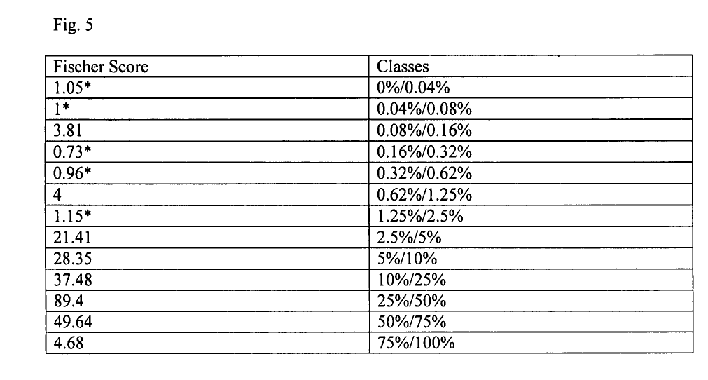

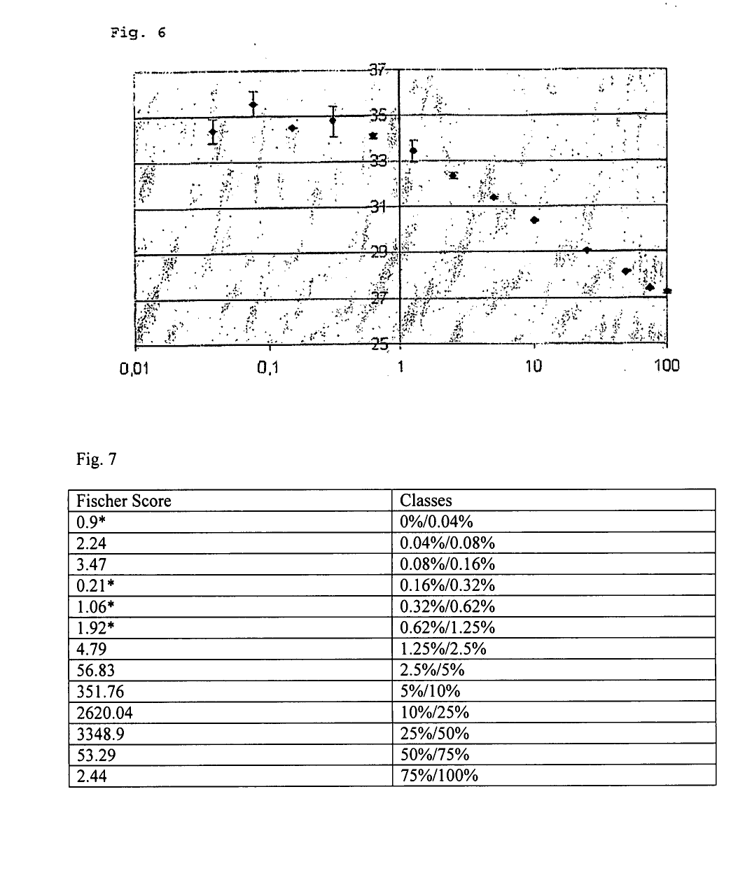

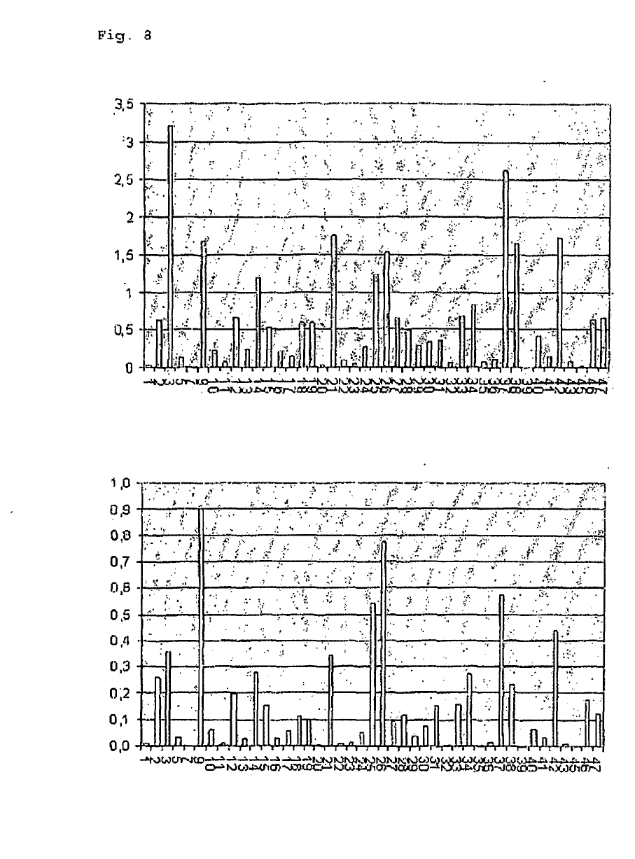

1. A method for quantifying methylated DNA, characterized in that the following steps are conducted:
a) the DNA is converted with at least one methylation-specific restriction enzyme,
b) a real-time PCR is conducted, wherein the sequence to be investigated is only amplified if it has not been cleaved previously in step a),
c) the fraction of methylated DNA in the original specimen is calculated from the signal of the sequence to be investigated and the signal of a reference measurement.

2. The method according to claim 1, further characterized in that the reference measurement is conducted by means of a reference fragment which is found in the vicinity of the sequence to be analyzed.

3. The method according to claim 2, further characterized in that the amplifications of the sequence to be analyzed and of the reference fragment take place simultaneously in one reaction vessel.

4. The method according to claim 2, further characterized in that the amplifications of the sequence to be analyzed and of the reference fragment take place in different vessels.

5. The method for quantifying methylated DNA is characterized in that the following steps are conducted:
a) the isolated DNA is divided into two equal portions,
b) the first portion of the DNA is reacted with a methylation-specific restriction enzyme, while the other portion remains untreated,
c) the DNA of both portions is amplified by means of a real-time PCR, wherein fragments will only be formed in the first portion of the specimen, if the DNA has not been cleaved previously,
d) the fractions of methylated and unmethylated DNA in the original specimen are calculated from the signals of the two portions.

6. The method according to claim 1, further characterized in that the biological specimen is embedded in paraffin.

7. The method according to claim 1, further characterized in that the sequence to be amplified bears several restriction cleavage sites.

8. The method according to claim 7, further characterized in that the sequence to be amplified bears several restriction cleavage sites for the same enzyme.

9. The method according to claim 7, further characterized in that the sequence to be amplified bears restriction cleavage sites for different enzymes.

10. The method according to claim 1, further characterized in that the degree of methylation M is calculated according to the following formula:
M=E^{-ΔCt}, wherein E is the PCR efficiency and −ΔΔCt is the difference between the threshold value of the restricted fragment that is investigated and the reference fragment minus the systematic difference ΔCt.

11. The method according to claim 5, further characterized in that the degree of methylation M is calculated according to the following formula: M=E^{-ΔCt}, wherein E is the PCR efficiency and −ΔCt is the difference between the threshold value of the reference fragment and the threshold value of the cleaved specimen.

12. The method according to claim 1, further characterized in that a control is carried out, with which an examination can be made of whether the restriction is complete.

13. The method according to claim 12, further characterized in that unmethylated DNA is utilized as a control.

14. The method according to claim 13, further characterized in that another restriction enzyme is utilized for the control, which cleaves independent of methylation within the fragment to be amplified.

15. The method according to claim 14, further characterized in that an isoschizomer is utilized.

16. The method according to claim 12, further characterized in that a control gene is analyzed, which is present unmethylated.

17. The method according to claim 1, further characterized in that the quantification is carried out for the diagnosis of cancer disorders or other diseases associated with a change in the methylation state.

18. The method according to claim 1, further characterized in that the quantification is carried out for predicting undesired drug effects and for distinguishing cell types or tissues or for investigating cell differentiation.

19. The method according to claim 1, further characterized in that a sensitive quantification is carried out.

20. The method according to claim 19, further characterized in that the fraction of the methylation state to be detected amounts to less than 10%.

21. The method according to claim 20, further characterized in that the fraction of the methylation state to be detected amounts to less than 5%.

22. The method according to claim 21, further characterized in that the fraction of the methylation state to be detected amounts to less than 1%.

23. A kit for conducting the method of claim 1, which is comprised of at least one restriction enzyme, two primers, a polymerase and a specific real-time probe or a non-sequence-specific intercalating fluorescent dye, as well as other optional reagents necessary for a PCR.
